(19) United States
(12) Patent Application Publication
(10) Pub. No.: US 2026/0263699 A1
(43) Pub. Date: Sep. 10, 2026

(54) AUTO-DISABLE SYRINGE

(71) Applicants: Richard J. Caizza, Union City, NJ (US); Connor Caizza, Tinton Falls, NJ (US)

(72) Inventors: Richard J. Caizza, Union City, NJ (US); Connor Caizza, Tinton Falls, NJ (US)

(21) Appl. No.: 19/658,519

(22) Filed: Apr. 25, 2026

(51) Int. Cl. A61M 5/315; A61M 5/31

(52) U.S. Cl. CPC A61M 5/31501; A61M 5/3129; A61M 5/31515; A61M 5/31548; A61M 5/3158; A61M 2005/31508; A61M 2202/0007; A61M 2202/04; A61M 2205/273

(57) ABSTRACT

A fixed-dose auto-disable syringe and locking clip therefor, the syringe having a barrel with an axial fluid chamber length reduced by approximately 20% to 40%, achieved by removing a non-functional proximal barrel portion while preserving the inner diameter, distal geometry, and fill volume. The auto-disable mechanism comprises a stamped stainless steel open-arc locking clip carrying three inwardly directed tabs evenly spaced at approximately 85 degree intervals that engage plunger rod serrations and maintain coaxial alignment of plunger rod with barrel axis, and two outwardly directed tabs that embed in the barrel inner wall to prevent reuse. A circumferential gap of approximately 104 degrees permits lateral placement of clip onto the plunger rod at predetermined dose-setting axial position. A frangible reduced-diameter section in plunger rod fractures at controlled force upon any reuse attempt, rendering the syringe permanently unusable. An alternative embodiment provides an integrally moulded plunger rod and piston.

### FIELD OF THE INVENTION

The present invention relates to auto-disable hypodermic syringes for fixed-dose immunization, and more particularly to an auto-disable syringe for preventing reuse of the syringe following delivery of a fixed vaccine dose.

### BACKGROUND OF THE DISCLOSURE

The reuse of hypodermic syringes intended for single use is a major contributor to the transmission of blood-borne pathogens including human immunodeficiency virus (HIV), hepatitis B virus, and hepatitis C virus. The World Health Organization (WHO), UNICEF, the Pan American Health Organization (PAHO), and the International Federation of Red Cross and Red Crescent Societies have collectively identified the auto-disable (AD) syringe as the preferred device for all fixed-dose immunization programs. Auto-disable syringes are designed to be automatically and permanently rendered incapable of reuse upon delivery of the intended fixed dose of vaccine, without requiring any additional action by the user. The international standard governing AD syringes for fixed-dose immunization is ISO 7886-3:2020.

Mass vaccination campaigns are primarily conducted in low- and middle-income countries that face constraints in medical waste management. In these settings, used syringes are typically disposed of by incineration. The volume and mass of syringe waste directly affects the cost, time, and environmental impact of waste disposal. Reduction in the physical size of the AD syringe—including its barrel length, packaging volume, and shipping weight—has a compounding positive effect on the economics and environmental footprint of large-scale immunization programs.

Conventional auto-disable syringes, including commercially available fixed-dose immunization syringes, include a syringe barrel whose total axial length substantially exceeds the length required to perform two functions: (i) to contain the nominal dose volume of fluid between the piston and the needle tip; and (ii) to provide the plunger stroke length needed to actuate the auto-disable mechanism at the completion of the injection. A significant portion of the proximal end of the barrel in conventional designs serves no functional purpose with respect to fluid containment, plunger stroke, or auto-disable activation. This non-functional proximal barrel section arises historically from the manufacturing and tooling conventions of conventional disposable syringe design, and has been carried forward into AD syringe designs without re-examination of whether the proximal barrel length is necessary.

The non-functional proximal barrel length imposes the following technical and commercial disadvantages, each compounded by the scale of production and use in global immunization campaigns:

Excess polymer mass per syringe: the non-functional proximal barrel section contributes polymer volume that must be injection-moulded, increasing raw material consumption per unit. At production volumes of hundreds of millions of units, this represents a substantial and avoidable raw material cost and resource consumption.

Extended mould cycle time: the greater polymer mass of a longer barrel requires a longer cooling time per injection moulding cycle, reducing throughput per mould cavity and increasing energy consumption per unit produced.

Larger packaging footprint: a longer barrel requires a longer primary packaging pouch, a larger shelf carton, and a larger case carton. These packaging increments accumulate to a significant increase in shipping volume and weight per dose, raising logistics costs for both manufacturers and procurement agencies.

Greater post-use volume: the larger physical size of the used syringe limits the number of syringes that fit safely into a sharps protective container thus requiring additional sharps containers that are expensive and increases the volume of medical waste requiring disposal, increasing the burden on local waste infrastructure, fuel cost of incineration, and volume of residual ash.

Reduced one-handed operability and increased patient anxiety: a longer barrel is more difficult for a healthcare worker to operate with one hand, and more visible to the patient. Patient visibility of the syringe is a recognised contributor to injection anxiety clinically known as trypanophobia, affecting approximately 10% of the general population with higher prevalence in paediatric patients—which can lead to vaccine avoidance and non-compliance with immunization schedules.

There exists, therefore, a long-felt need in the art for an auto-disable syringe in which the barrel length is reduced by elimination of the non-functional proximal section, while fully preserving the nominal fill volume, the plunger stroke required for auto-disable actuation, and all performance requirements of ISO 7886-3:2020. This need has been recognised by the governing international standard itself: ISO 7886-3:2020, Clause 5(c) expressly requires that the barrel length shall be designed to enable accurate dosage while avoiding unnecessary waste of vaccine. Despite this express requirement, no prior art has addressed it by the specific structural means of the present invention.

U.S. Pat. No. 5,562,623 (Univec, Inc.) discloses a single-use syringe in which a radially resilient locking spring clip of stamped stainless steel is mounted on a plunger rod having frusto-conical ratchet teeth. A characteristic feature of this prior art clip is that it deliberately displaces the plunger rod off-axis within the barrel during operation: the asymmetric engagement of the clip against the plunger rod on one side only creates a radial side-load that pushes the plunger rod toward the opposite barrel wall. The '623 patent discloses that this off-axis operation was an essential feature enabling miniaturisation of the device to small volumes. Furthermore, the '623 patent does not disclose or suggest any modification to the axial length of the syringe barrel; the barrel geometry is conventional and includes the non-functional proximal barrel section.

U.S. Pat. No. 6,533,756 (Univec, Inc.) discloses a single-use syringe in which a barbed metal clip is mounted on a serrated vane of an X-shaped plunger rod. This design is specific to a plunger rod of X-shaped cross-section having discrete longitudinal vanes. The '756 patent does not disclose barrel length reduction.

U.S. Pat. No. 9,205,205 (Becton Dickinson) discloses a reduced-size auto-disable syringe in which the locking mechanism comprises a plastic element with the plunger rod. While the '205 patent describes a syringe of reduced overall size, the size reduction is achieved by simultaneously reducing both the barrel axial length and the barrel inner diameter from a substantially different baseline design: the reference syringe of the '205 patent has an inner diameter of approximately 8.73 mm and a total length of approximately 51.40 mm, and the disclosed reduced-size embodiment has an inner diameter of approximately 4.70 mm and a length of approximately 47.82 mm. This represents a comprehensive redesign with both dimensional parameters changed simultaneously. The '205 patent does not specifically disclose the targeted removal of only the non-functional proximal portion of an existing commercial syringe barrel while preserving all other barrel dimensions.

Chinese Patent No. CN102488948B discloses a self-destruction syringe with a molten medicine district—a device designed for reconstitution and injection of lyophilised powder preparations. The inventive concept is a smooth zone on the plunger rod within which a polymer spring locking element can be reciprocated to dissolve the powder without triggering the auto-disable mechanism prematurely. The locking element is a polymer spring ring. CN102488948B does not disclose barrel length reduction.

European Patent Application No. EP2168622A1 (Wuxi Xushou Medical Appliances) discloses a self-destructive syringe also directed to the reconstitution of powder drugs. The non-return element is an elastic steel sheet mounted within a slot in the plunger rod itself. EP2168622A1 does not disclose barrel length reduction.

Accordingly, there exists a need in the art for an auto-disable syringe for fixed-dose immunization that reduces material consumption, moulding cycle time, packaging volume, and post-use waste relative to conventional designs, while fully preserving the nominal fill volume, auto-disable reliability, and consistent injection force throughout the delivery stroke.

### SUMMARY OF THE DISCLOSURE

It is an objective of the present invention to provide a fixed-dose auto-disable syringe having a barrel of reduced axial length achieved by removal of a non-functional proximal barrel portion while preserving all other functional dimensions.

It is another objective of the present invention to provide the syringe as above wherein an auto-disable mechanism comprises a locking clip mounted on a serrated plunger rod.

It is yet another objective of the present invention to provide a method of manufacturing the syringe as above by laterally placing the locking clip onto the serrated plunger rod at a dose-setting axial position and inserting the assembly into the reduced-length barrel.

It is a further objective of the present invention to provide an alternative embodiment of the syringe as above wherein the piston is integrally moulded with the plunger rod as a single component.

It is another objective of the present invention to reduce barrel polymer mass, raw material consumption, and moulding cycle time relative to conventional auto-disable syringe designs.

It is yet another objective of the present invention to reduce packaging volume, shipping weight, and post-use incineration waste, of particular benefit in resource-limited settings.

It is another objective of the present invention to provide consistent injection force and to eliminate asymmetric barrel loading during the injection stroke.

It is yet another objective of the present invention to provide a fixed-dose auto-disable syringe that fully conforms to ISO 7886-3:2020.

These and other objectives and advantages of the present invention will become apparent from the following detailed description, taken in conjunction with the accompanying drawings.

### BRIEF DESCRIPTION OF THE DRAWINGS

The accompanying drawings, which are incorporated in and form a part of this specification, illustrate preferred embodiments of the invention and, together with the description, serve to explain the principles of the invention. In the drawings, like reference numbers identify like elements.

FIG. 1 is an exploded perspective view of an auto-disable syringe according to an embodiment of the present invention.

FIG. 2A and FIG. 2B depict a comparison of a conventional reference auto-disable syringe and an auto-disable syringe of the present invention, illustrating the reduction in total barrel length and the elimination of the non-functional proximal barrel portion.

FIGS. 3A and 3B depict syringe assembly steps.

FIGS. 4A and 4B depict steps to use assembled syringe of the present invention.

FIG. 5 is a perspective view of a locking clip depicting inward tabs and outward tabs.

FIG. 6 is a top view of the locking clip showing the three-dimensional open-arc body formed from a stamped metal blank.

FIG. 7 is a bottom view of the locking clip showing outer diameter; circumferential gap angle; gap opening width; angular separation between outward tabs D and E; and arc span of the inward tab region.

FIG. 8 is a cross-sectional view through section 8-8 of FIG. 6, illustrating inward tabs A, B, and C inclined at angle e relative to the longitudinal axis of the plunger rod.

FIG. 9 is a cross-sectional view through section 9-9 of FIG. 7, illustrating outward tabs D and E inclined at angle f relative to the longitudinal axis of the plunger rod.

FIG. 10 is a perspective view of an alternative plunger rod embodiment having an integrally molded piston stopper.

FIG. 11 is an isometric view of a barrel of the present invention showing the external form of the shortened barrel body, barrel flange, and needle hub.

FIG. 12 is an orthographic side view of the barrel.

FIG. 13 is a cross-sectional view of the barrel.

### DETAILED DESCRIPTION OF THE DISCLOSURE

The following detailed description is set forth to enable any person having ordinary skill in the art of disposable syringe design and manufacture to make and use the invention. Specific details of dimensions, materials, angular relationships, and operating forces are provided with respect to the preferred embodiments to illustrate the best mode known to the inventors. These specific details are not to be understood as limiting the scope of the invention as defined by the claims. Other embodiments will be apparent to persons skilled in the art without departing from the spirit and scope of the invention as claimed.

All numerical values stated in this specification, including linear dimensions, angular inclination values, arc measurements, force values, tolerance ranges, and diameter specifications, are exemplary values associated with preferred embodiments. These values are provided to enable a person of ordinary skill in the relevant art to make and use the invention and to illustrate the preferred implementation of the inventive concept. No stated dimension or value is to be construed as an absolute limitation on the scope of the invention unless that value is expressly recited as a claim limitation. The invention encompasses equivalent implementations in which one or more stated values differ from the preferred exemplary values, provided that the functional result achieved is substantially equivalent to that described herein. Variations attributable to manufacturing tolerances, material property differences, scale changes, and application-specific design requirements are within the scope of the invention.

As used herein, the terms “proximal” and “distal” refer to the orientation of the syringe in normal use: the proximal end is the end towards the user's hand (the plunger end), and the distal end is the end towards the patient (the needle end). The terms “proximal movement” and “distal movement” refer to axial movement of the plunger rod towards the proximal end (withdrawal, for aspiration) and towards the distal end (advancement, for injection) respectively.

As used herein, the term “reference syringe” or “reference auto-disable syringe” refers to a commercially available fixed-dose auto-disable syringe of the same nominal fill volume as the syringe of the present invention, whose barrel and plunger geometry forms the baseline from which the present invention departs. In the preferred embodiment described herein, the reference syringe has a total barrel length of 82.10 mm, a fluid chamber axial length of 72.80 mm, and an inner barrel diameter of 4.70 mm. The design intent of the present invention is not limited to improvement of this specific reference syringe and may be applied to any conventional auto-disable syringe in which the barrel axial length exceeds the functional minimum required for dose delivery and auto-disable actuation.

The auto-disable syringe of the present invention conforms in all respects to ISO 7886-3:2020. All references to ISO 7886-3:2020 herein are to the second edition, dated May 2020.

The following reference numerals are used consistently throughout this description and the accompanying drawings:
101—Needle (hypodermic cannula)
102—Barrel (syringe body)
103—Piston (plunger stopper)
104—Serrated plunger rod
105—Auto-disable mechanism
106—Needle cap (needle shield)
107—Fluid bottle (vaccine vial or ampoule; external to the invention)
108—Barrel flange (finger grip)
109—Plunger rod flange (thumb button)

With reference to the auto-disable mechanism 105, the individual tabs are designated: Tab A—first inwardly directed tab; Tab B—second inwardly directed tab; Tab C—third inwardly directed tab; Tab D—first outwardly directed tab; Tab E—second outwardly directed tab.

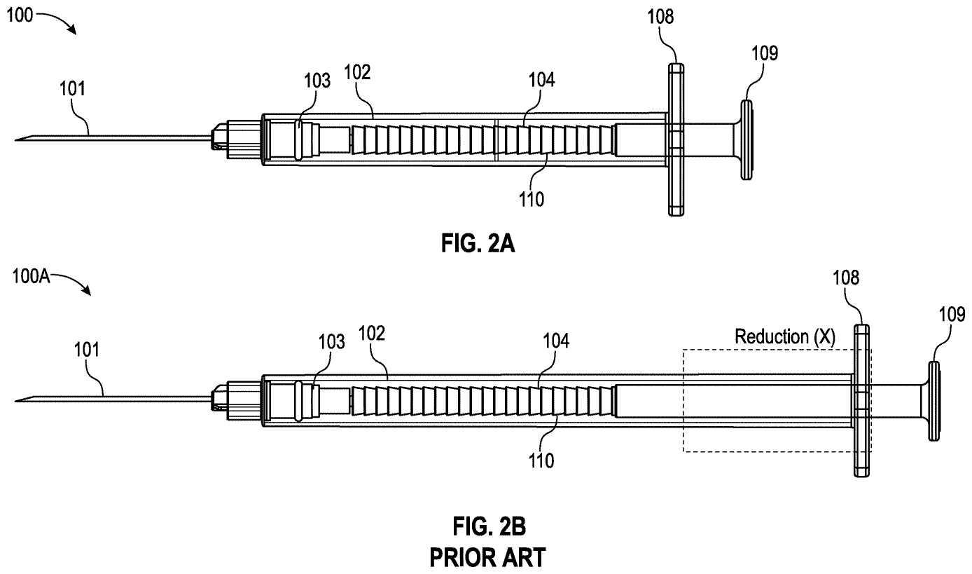

Referring to FIG. 2A, an auto-disable syringe 100 of the present invention is shown in side-by-side comparison with a conventional reference auto-disable syringe 100A of the same nominal fill volume as shown in FIG. 2B. Typically, the conventional reference syringe 100A has a total barrel length of 82.10 mm, of which approximately 22.10 mm at the proximal end does not contribute to the fluid chamber, the plunger stroke, or the auto-disable actuation stroke—this is the non-functional proximal barrel section that the present invention eliminates. In an exemplary embodiment, the syringe 100 of the present invention has a total barrel length of 60.00 mm, achieved by removing that 22.10 mm non-functional proximal section. All other external and internal dimensions and geometries of the barrel are preserved.

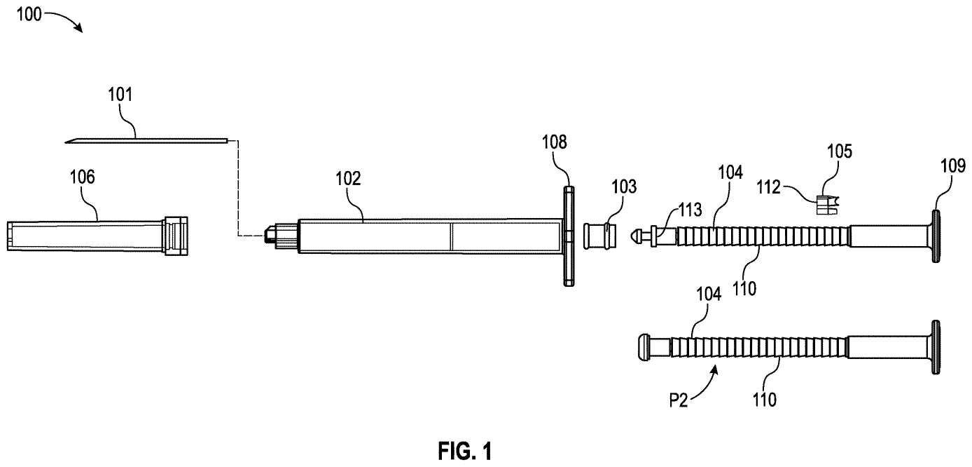

Referring now to FIG. 1, the auto-disable syringe 100 of the present invention is shown in an exploded perspective view. The assembly consists of: a needle 101 fixed within a needle hub at a distal end of a barrel 102; the barrel 102 having the barrel flange 108 at its proximal end; a piston 103 forming a fluid-tight seal against the inner wall of the barrel 102; a serrated plunger rod 104 carrying the piston 103 at its distal end and a plunger rod flange 109 at its proximal end; and an auto-disable mechanism 105 mounted on the serrated plunger rod 104 at a dose-setting axial position. A needle cap 106 covers the needle 101 prior to use. The auto-disable mechanism 105 is also referred to as a “locking clip” or “clip”.

Referring to FIGS. 11, 12, and 13, the barrel 102 is a hollow generally cylindrical body injection-moulded from transparent medical-grade polypropylene. Transparency is required by ISO 7886-3:2020, Clause 5(a), to allow the graduated scale markings and the piston position to be observed during filling. The polypropylene material is biocompatible in accordance with ISO 10993-1. The barrel 102 is provided at its proximal end with the barrel flange 108, extending radially outward in a plane perpendicular to the barrel axis, providing the contact surface for the index and middle fingers during one-handed operation.

At the distal end, the barrel 102 carries a needle hub formed integrally with the barrel body during injection moulding. The needle 101 is a stainless steel hypodermic cannula bonded into the needle hub, forming an integrated needle assembly. The distal geometry, taper angle, and needle hub interface of the barrel 102 are identical to those of the reference syringe; no modification to the distal barrel geometry has been made. The inner surface of the barrel 102 is lubricated with a silicone lubricant to reduce initial breakaway force and mean injection force, in accordance with ISO 7886-1:2017, Clause 7. The barrel graduation scale comprises two markings only: the zero graduation line and the nominal capacity graduation line, in accordance with ISO 7886-3:2020, Clause 9.1.

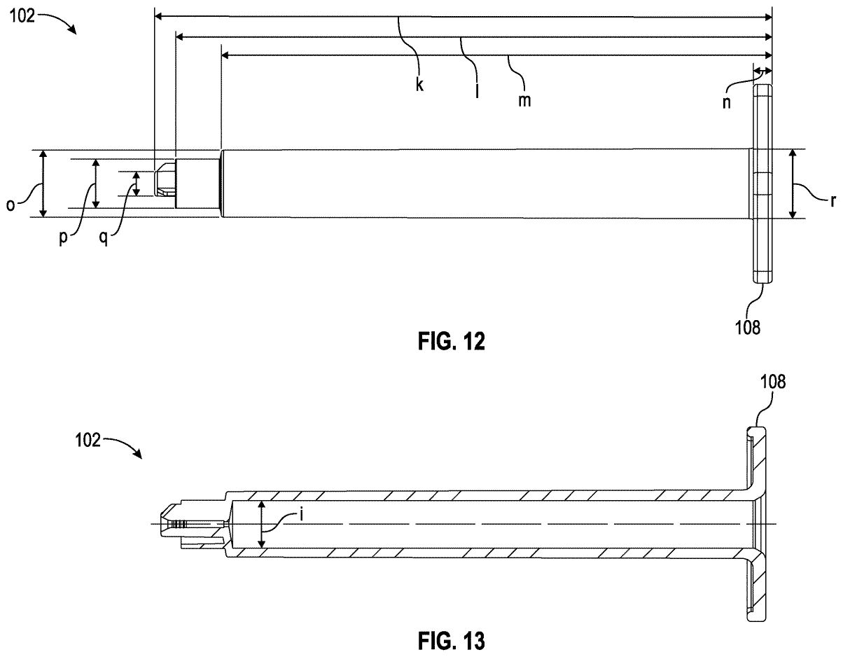

Referring to FIG. 12, the total axial length of the barrel 102 in the preferred embodiment is k=60.00 mm, measured from the proximal face of the barrel flange 108 to the distal tip of the needle hub. The axial length of the fluid chamber—defined as the distance from the fully distal position of the piston face to the internal datum of the needle hub is m=53.54 mm.

These dimensions are to be compared with the corresponding dimensions of the reference syringe: total barrel length of 82.10 mm and fluid chamber axial length of 72.80 mm. The reduction in total barrel length is:

$$( 82.1 mm - 60. mm ) \div 82.1 mm \times 100 = 26.9 \% , approximately 27 \% .$$

The reduction in fluid chamber axial length is:

$$( 72.8 mm - 53.54 mm ) \div 72.8 mm \times 100 = 26.5 \% , approximately 27 \% .$$

Both the total barrel length and the fluid chamber axial length are reduced by approximately 27% in the preferred embodiment. Barrel length reductions in the range of 20% to 40% relative to a reference syringe of the same nominal fill volume are contemplated within the scope of the invention.

Referring to FIG. 13, the inner diameter of the barrel 102 is i=4.70±0.10 mm, identical to the inner diameter of the reference syringe. The outer diameter r of the barrel 102 is 6.50 mm and the barrel wall thickness is approximately 0.90 mm. Critically, no change is made to the inner diameter, the distal geometry, or the needle tip interface relative to the reference design. The entire dimensional reduction is confined to the proximal barrel section.

The fluid chamber length-to-inner-diameter ratio for the barrel 102 of the present invention is:

$$53.54 mm \div 4.7 mm = approximately 11.4 : 1.$$

This compares with the corresponding ratio for the reference syringe of approximately 15.5:1. The preferred range for this ratio in the present invention is 8:1 to 14:1, which accommodates the majority of fixed-dose vaccine volumes of 0.5 mL or less while minimising overall barrel length.

Table 1 below provides a dimensional comparison of the barrel 102 of the present invention, the reference syringe, and the prior art reduced-size syringe of U.S. Pat. No. 9,205,205.

TABLE 1
Dimensional comparison of barrel 102 of the present
invention 100 with reference syringe and prior art.

| | Reference | | | | U.S. Pat. No. | |
|---|---|---|---|---|---|---|
| Parameter | syringe | | Present invention | | 9,205,205 | |
| Total barrel length | 82.10 | mm | 60.00 | mm (−27%) | 47.82 | mm |
| Fluid chamber length | 72.80 | mm | 53.54 | mm (−26.5%) | ~47.82 | mm |

| | | | | | |
|---|---|---|---|---|---|
| Inner diameter (ID) | 4.70 | mm | 4.70 | mm (unchanged) | 4.70 mm (reduced |
| | | | | | from 8.73 mm) |

| | | | | | |
|---|---|---|---|---|---|
| Outer diameter (OD) | 7.00 | mm | 6.50 mm | 5.90 | mm |
| Wall thickness | ~1.15 | mm | ~0.90 mm | ~0.61 | mm |

| | | | |
|---|---|---|---|
| L:D ratio (fluid | ~15.5:1 | ~11.4:1 | ~10.2:1 |
| chamber) | | | |
| Diameter change | N/A | None - ID identical | Reduced from 8.73 |
| from baseline | | to reference | mm to 4.70 mm |
| Approach to size | N/A | Proximal-only | Simultaneous reduction of length |
| reduction | | removal; all other | and diameter from a different |
| | | dimensions preserved | larger baseline design |

As Table 1 shows, the approach of the present invention is structurally and conceptually distinct from that of U.S. Pat. No. 9,205,205. The Becton Dickinson '205 patent achieved size reduction by redesigning the barrel from a different, larger-calibre starting point, reducing both the barrel length and the inner diameter simultaneously. The present invention takes a targeted approach: starting from a commercially established reference syringe design with a 4.70 mm inner diameter, only the non-functional proximal barrel section is removed, and all other dimensions are preserved unchanged. This distinction is both structurally specific and functionally significant.

ISO 7886-3:2020, Clause 5(c) requires that: “The length of the barrel shall allow the expulsion of any air bubbles without removal of the plunger stopper and still inject nominal capacity. The length of the barrel shall be designed to enable accurate dosage while avoiding unnecessary waste of vaccine.”

The barrel 102 of the present invention satisfies both requirements of Clause 5(c). The fluid chamber length of 53.54 mm provides sufficient stroke length for the piston to travel from the fully proximal fill position to the fully distal injection position, expelling the full nominal dose volume. Air bubbles may be expelled by briefly advancing the plunger rod prior to injection without removing the piston from the barrel. The barrel length has been minimised to eliminate the proximal section that was “unnecessary” within the meaning of Clause 5(c), as that proximal section contributed nothing to air expulsion, dose delivery, or auto-disable actuation.

The maximum usable capacity of the barrel 102 does not exceed the nominal capacity by more than 25%, in accordance with ISO 7886-3:2020, Clause 10.1. This is ensured by the combined action of the barrel length and the axial position of the locking clip 105 on the plunger rod 104 as set at manufacture.

In addition to the manufacturing and environmental advantages described above, the reduced barrel length of the present invention provides an unexpected clinical benefit identified during prototype evaluation. Healthcare workers found that the shortened syringe 100 is substantially easier to conceal within the palm of one hand during the approach to injection. The palm-concealment technique—in which the syringe 100 is held loosely within the closed fingers of the healthcare worker's hand and is not visibly presented to the patient until the moment of needle contact—was found to be significantly more reliably achievable with the shortened barrel than with a conventional barrel of standard length.

This benefit is of particular clinical significance in the administration of vaccines to paediatric patients and to adult patients with trypanophobia, which is the clinical term for an intense fear of hypodermic injection needles. Trypanophobia is estimated to affect approximately 10% of the general population and is associated with elevated levels of procedure-related anxiety, increased risk of vasovagal syncope, and in severe cases, avoidance of medically necessary vaccinations and injections. The ability to conceal the syringe 100 within the healthcare worker's palm during approach to injection reduces the patient's visual exposure to the device and can meaningfully reduce procedural anxiety and improve compliance with immunization schedules.

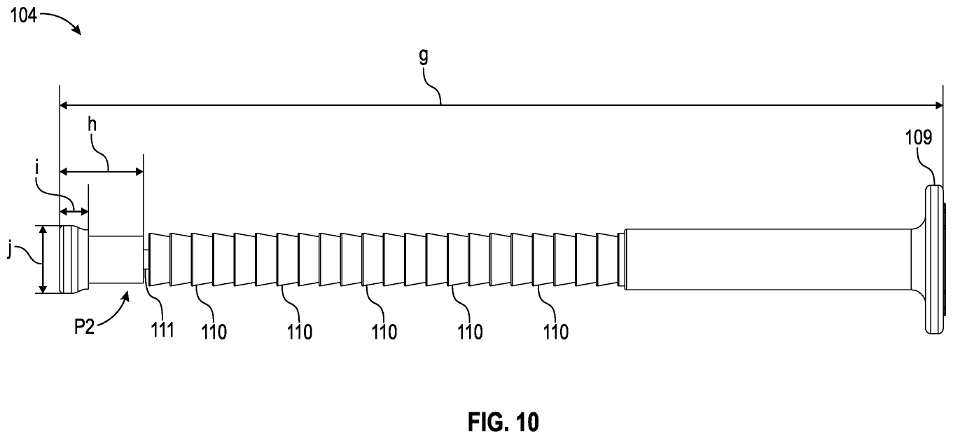

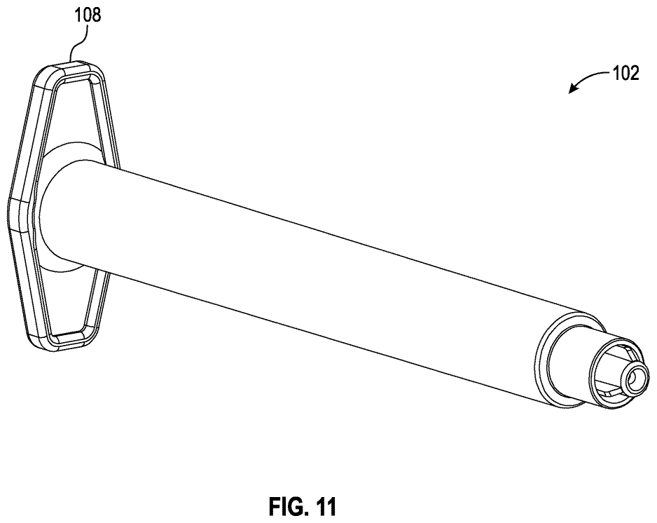

Referring to FIG. 10, the serrated plunger rod 104 is a generally cylindrical rod formed from injection-moulded polypropylene modified by the incorporation of an embrittlement additive. The embrittlement additive reduces the elongation-at-break of the polypropylene, ensuring that a frangible section 111 of the plunger rod fractures cleanly under axial tensile load without undergoing excessive plastic deformation that might allow the plunger rod to remain functionally intact after an attempted reuse.

The overall length g of the plunger rod 104 is 59.9 mm in the primary embodiment, selected to be commensurate with the reduced barrel length of 60.00 mm so that the plunger rod flange 109 is accessible for thumb operation throughout the full stroke, and so that the minimum plunger projection above the barrel flange 108 surface meets the preferred minimum of 8 mm specified in ISO 7886-3:2020, Clause 11.1, when the piston 103 is at the zero graduation line.

At its proximal end, the plunger rod 104 carries the plunger rod flange 109 a flat disc-shaped thumb contact element providing a surface for the user to apply a controlled distally directed force during injection. At its distal end, the plunger rod 104 supports the piston 103. In the primary embodiment, the piston 103 is a separate component made from a rubber or thermoplastic elastomer material, attached to the distal end of the plunger rod 104 by a mechanical interference fit.

The exterior surface of the cylindrical portion of the plunger rod 104 comprises a plurality of serrations (ratchet teeth) 110 that cooperate with the inward tabs A, B, C of the locking clip 105. In a preferred embodiment, the serrations 110 are formed with a pitch of 1.50 mm. Each serration 110 has a crest outer diameter of 4.25 mm and a root outer diameter of 3.70±0.10 mm, giving a tooth height of approximately 0.275 mm per side.

The serration profile 110 has an asymmetric cross-sectional form with an included angle of 96 degrees. The proximal face of each serration 110 is sloped, allowing the inward tabs A, B, C of the locking clip 105 to cam over the serration 110 during proximal withdrawal of the plunger rod (aspiration). The distal face of each serration 110 is substantially perpendicular to the longitudinal axis of the plunger rod 104, providing a positive mechanical engagement surface for the inward tabs A, B, C during distal advancement (injection) of the plunger rod 104, carrying the locking clip 105 distally with the plunger rod 104.

The root outer diameter of 3.70 mm is greater than the gap opening width of the locking clip 105 of 3.03 mm. This dimensional relationship ensures that after the locking clip 105 has been placed laterally onto the plunger rod 104 through the clip gap, the clip 105 cannot be accidentally dislodged from the plunger rod 104 by radial force during normal handling or assembly, since radial removal would require the clip gap to expand over the 3.70 mm root diameter, requiring deliberate application of force beyond the normal handling range.

A frangible reduced-diameter section 111 is formed on the plunger rod 104 at a position adjacent to the distal-most serration, in close proximity to the piston 103. In the preferred embodiment, the frangible section 111 has a diameter of 1.35±0.10 mm. The precise axial location of the frangible section 111 is 10.4 mm from the distal end P2 of the plunger rod 104 and 8 mm from the proximal face of the piston 103.

The purpose of the frangible section 111 is to provide a deliberate structural weak point in the plunger rod 104 that will fracture under axial tensile load if a user attempts to withdraw the plunger rod 104 after the auto-disable feature has been activated, thereby destroying the functionality of the syringe 100 and preventing any possibility of reuse.

The design of the frangible section 111 is calibrated to meet the performance requirements of ISO 7886-3:2020, Annex C. Clause C.3.1 requires that it shall not be possible to reuse the syringe 100 after applying a force of 100 Newtons at a speed of 100 mm/min on the plunger. The frangible section 111 of the plunger rod 104 is designed to fracture at an applied axial tensile force of between 38 Newtons and 55 Newtons a range well below the 100 Newton test threshold, ensuring that the syringe 100 is rendered unusable before the test force limit is reached. The selected fracture force range of 38 to 55 Newtons is also well above the forces normally applied by a user during filling and injection (typically less than 10 Newtons for a syringe 100 of this calibre and fill volume, as indicated by ISO 7886-3:2020, Annex B, Table B.1 for syringes of less than 2 mL nominal capacity), ensuring that the frangible section 111 does not fracture during normal intended use.

The location of the frangible section adjacent to the distal-most serration and proximal to the piston 103 means that when the plunger rod fractures, the proximal portion of the plunger rod 104 (including the plunger rod flange 109 and all serrations) separates from and leaves behind the piston 103 at the distal end of the barrel 102. The separated distal piston portion is physically too short to be grasped by a user and is confined within the barrel 102, making reuse of the syringe 100 structurally impossible.

The frangible section 111 also satisfies the back-pressure reuse prevention test of ISO 7886-3:2020, Annex C, Clause C.3.2, which requires that a back pressure of up to 300 kPa applied through the needle shall not be capable of driving the piston back through the barrel. Following auto-disable activation, the locking clip 105 physically blocks proximal movement of the plunger rod 104 and piston 103 under back pressure, and the geometry of the frangible section 111 and piston 103 does not permit back-pressure reversal.

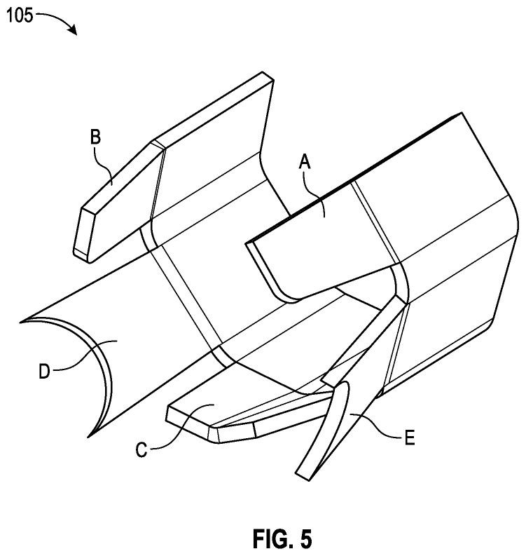

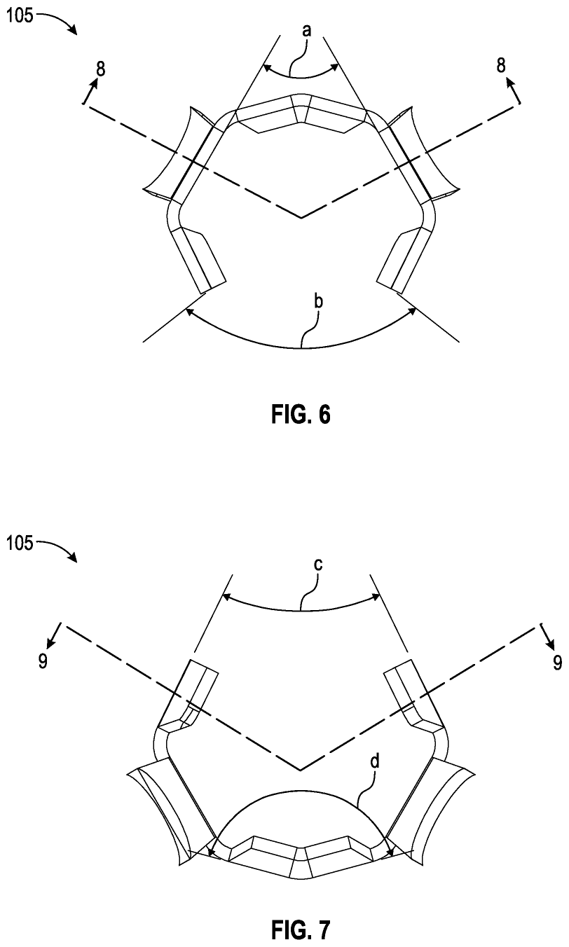

Referring to FIGS. 5 through 9, the locking clip 105 is formed from a single flat sheet of stainless steel in a preferred embodiment. The stainless steel material provides: (i) sufficient elastic spring force in the tab elements to allow the inward tabs A, B, C to flex over the serration crests during plunger withdrawal and to spring back to the engagement position at each serration root; (ii) sufficient hardness and sharpness at the contact tips of the outward tabs D and E to embed into the polypropylene inner wall of the barrel 102 under the embedding force generated during injection; and (iii) sufficient corrosion resistance for long-term storage in a sterile packaging environment.

A metal blank from which the clip is formed has a nominal thickness of 0.20 mm, within a tolerance range of +/−25%, i.e., between 0.15 mm and 0.25 mm. The blank thickness is selected to provide the appropriate combination of radial flexibility at the inward tabs (for smooth aspiration cam-over) and axial rigidity at the outward tabs (for reliable barrel wall embedding during injection). A thicker blank would require greater force for the inward tabs to flex over the serration crests, increasing the initial breakaway force of the syringe 100. A thinner blank would reduce the radial spring force of the outward tabs against the barrel inner wall, reducing the reuse prevention force and risking non-compliance with the 100 Newton requirement of ISO 7886-3:2020, Annex C.

Referring to FIG. 7, when viewed from above (in the plane perpendicular to the plunger rod axis), the locking clip 105 has a generally hexagonal open-arc plan form. The body of the clip 105 subtends an arc of approximately 256.2 degrees about the central axis of the plunger rod 104, leaving a circumferential gap of approximately 103.8 degrees (nominally 104 degrees). The outer diameter of the clip body is 5.42 mm. The gap opening width—measured as the straight-line distance between the two free edges of the clip body at the gap—is 3.03 mm.

The circumferential gap angle of 103.8 degrees and corresponding gap opening width of 3.03 mm are deliberate design choices selected to satisfy two simultaneous requirements that are in tension with each other:

First requirement—lateral loading during assembly: the gap must be large enough to permit the clip to be pressed onto the plunger rod 104 laterally—i.e., by pressing the gap over the rod from the side—without requiring axial threading of the clip over the end of the plunger rod. This lateral loading method is the intended and deliberate manufacturing process for the clip assembly step, enabling high-speed automated assembly. The clip gap of 3.03 mm is slightly less than the plunger rod root diameter of 3.70 mm; loading is accomplished by the radial spring flexibility of the stainless steel body, which allows the gap to spread from 3.03 mm to accommodate the root diameter during assembly, after which the body springs back to its natural gap width, retaining the clip on the rod.

Second requirement—retention on the plunger rod after loading: once placed on the plunger rod 104, the clip must not be accidentally dislodged during subsequent handling and assembly. With a gap opening width of 3.03 mm and a plunger rod root outer diameter of 3.70 mm, the clip cannot be radially dislodged from the plunger rod without a deliberate spreading force in excess of the forces encountered in normal handling. The dimensional relationship between the gap width and the rod diameter thus provides inherent retention of the clip on the rod without any additional retention feature.

Referring to FIG. 9 and to FIG. 5, the locking clip 105 carries three inwardly directed tabs designated A, B, and C. Each inward tab is formed by a combination of blank stamping and bending, projecting from the inner face of the clip body inwardly toward the axis of the plunger rod 104.

Referring to FIG. 9, each inward tab A, B, C is inclined at an angle f=10 degrees ±2 degrees relative to the longitudinal axis of the plunger rod 104, directed inwardly and distally (i.e., toward the needle end of the syringe 100). This inclination angle determines the cam-over characteristic of the tab against the sloped proximal face of the serrations during aspiration: the selected angle of 10 degrees+2 degrees is calibrated to provide smooth aspiration without perceptible ratcheting force to the user, while maintaining reliable clip engagement at each serration during the injection stroke.

The three inward tabs A, B, C are evenly spaced at approximately 85 degree intervals around the inner arc of the clip body. The arc coverage of 256.2 degrees divided by three tabs gives an inter-tab spacing of approximately 85.4 degrees between adjacent tabs, approximating equal distribution around three-quarters of the plunger rod circumference. Within the arc subtended by the clip body, this three-point distribution is the geometric arrangement that most closely approximates equal circumferential spacing.

The three-point symmetric arrangement of inward tabs A, B, C of the present invention distinguishes the clip from the prior art spring clip. In the prior art clip, two inward camming teeth are positioned on opposite sides of a clip body that subtends only approximately 200 degrees an arrangement that, by geometric necessity, produces a net lateral force on the plunger rod away from the clip opening, displacing the plunger rod off-axis within the barrel.

In the present invention, the three-point arrangement of inward tabs A, B, C produces three inward forces at approximately equal angular intervals around the plunger rod circumference, with the result that the net lateral force on the plunger rod is substantially zero. The plunger rod 104 is thereby maintained in coaxial alignment with the central axis of the barrel 102 during both aspiration and injection. This coaxial alignment:

Produces a symmetric, uniform distribution of the contact force between the piston 103 and the barrel inner wall, reducing localised friction and providing consistent injection force throughout the stroke.

Eliminates the asymmetric lateral loading of the barrel inner wall that characterises off-axis clip designs, reducing the risk of deformation or stress concentration in the barrel wall at the clip location.

Facilitates smooth, low-friction aspiration and injection without the plunger rod binding against the barrel wall, which is of particular importance for small-calibre barrels of 4 to 6 mm inner diameter where the lateral tolerance between the plunger rod and barrel inner wall is small.

Provides a symmetric engagement force between the three inward tabs and the plunger rod serrations, distributing the reaction force evenly around the plunger rod circumference and reducing the risk of plastic deformation or wear of any single serration tooth.

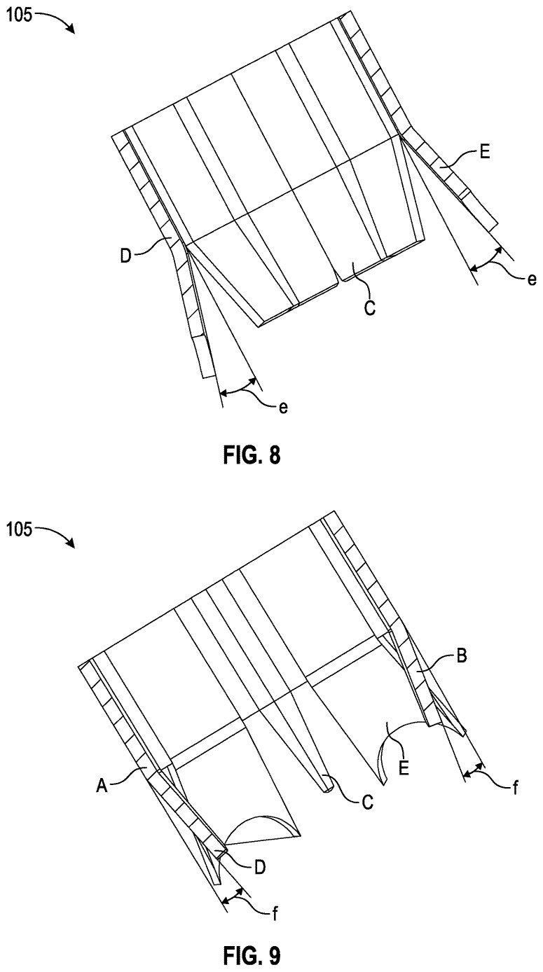

Referring to FIG. 8 and to FIG. 5, the locking clip 105 carries two outwardly directed tabs designated D and E. Each outward tab is formed by a combination of blank stamping and bending, projecting from the outer face of the clip body outwardly away from the axis of the plunger rod 104. Inward tab C is also visible in FIG. 8 at the centre of the clip body, confirming the positional relationship between the inward and outward tabs as viewed from this perspective.

Referring to FIG. 8, each outward tab D, E is inclined at an angle e=15 degrees ±2 degrees relative to the longitudinal axis of the plunger rod, directed outwardly and proximally (i.e., toward the plunger end of the syringe 100). This proximal inclination means that the tab tip points toward the proximal end of the barrel—the direction from which reuse force would be applied—so that any proximally directed force on the clip drives the tab tips more firmly into the barrel inner wall, increasing the resistance to withdrawal. The 15 degree ±2 degree inclination angle is selected to provide: (i) sufficiently low interference with the barrel inner wall during distal clip travel (injection stroke), allowing the clip to travel distally without the outward tabs causing undue drag; and (ii) sufficiently high embedding force against the barrel inner wall upon proximal loading (reuse attempt) to resist withdrawal under up to 100 Newtons as required by ISO 7886-3:2020, Annex C. The inclination angle e of outward tabs D and E, as marked in FIG. 8, is distinct from the inclination angle f of the inward tabs A, B, C as marked in FIG. 9.

The two outward tabs D and E are positioned on the clip body with an angular separation of 52 degrees between them. Both tabs are located within a 150 degree arc sector on the side of the clip body opposite to the circumferential gap. The two-point barrel engagement of tabs D and E provides a stable, non-rocking contact with the barrel inner wall, generating the embedding force and friction needed for reuse prevention.

The stainless steel material and the 15 degree inclination of the outward tabs D and E are selected such that the embedding force of the outward tabs into the polypropylene barrel inner wall, when the clip is at its full distal position after injection, exceeds the tensile strength of the frangible section 111 of the plunger rod (38 to 55 Newtons). This ensures that upon a reuse attempt, the plunger rod will fracture before the clip can be displaced proximally from its locked position, in full compliance with the anti-reuse requirement of ISO 7886-3:2020, Annex C.

Referring to FIG. 10, an alternative embodiment of the plunger rod 104 is provided in which the piston is integrally formed as a unitary moulded component with the plunger rod body, rather than as a separate rubber stopper element. In this alternative embodiment, the overall length of the integrated plunger rod and piston assembly is g=62.15 mm. The integrated piston portion at the distal end has an outer diameter of 4.75 mm, providing a slight interference fit with the 4.70 mm inner diameter of the barrel 102 to achieve the fluid-tight sliding seal required for aspiration and injection. The interference fit of 0.05 mm nominal is sufficient to maintain a gas-tight and liquid-tight seal against the barrel inner wall throughout the full injection stroke, as required by ISO 7886-3:2020, Clauses 13.3 and 13.4.

The serrated region 111, frangible section 111, and plunger rod flange 109 of the alternative plunger rod are identical in all respects to the aforementioned primary embodiment. The frangible section 111 has the same diameter of h=1.35 mm, providing equivalent fail-safe anti-reuse protection. The primary advantage of this alternative embodiment is the elimination of the separate piston component, reducing the total number of syringe assembly components from five to four and simplifying manufacturing assembly in high-volume production.

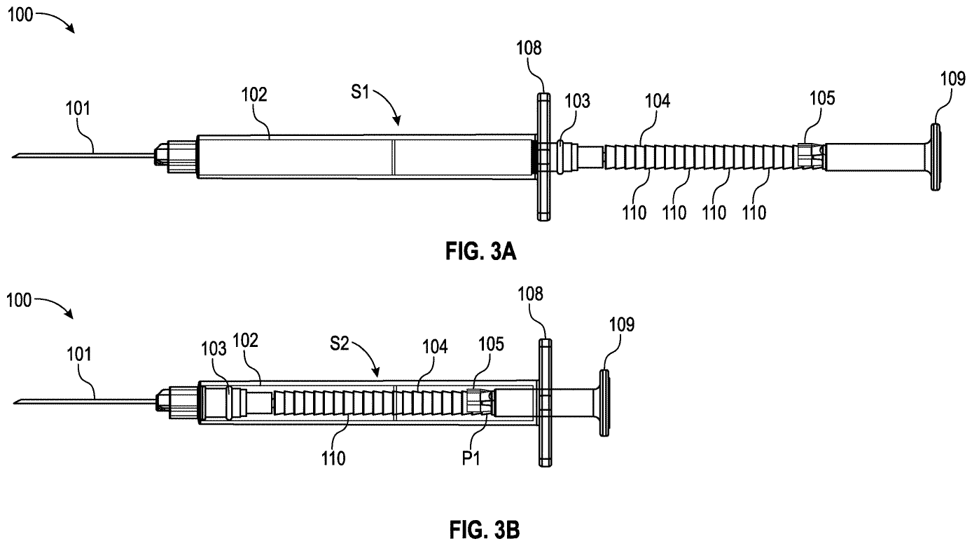

Referring to FIG. 3A, a first step S1 of the assembly process comprises positioning the locking clip 105 on the serrated plunger rod 104 at a predetermined axial position corresponding to the dose-setting position for the nominal fill volume of the syringe 100 being assembled.

The clip 105 is placed onto the plunger rod 104 by pressing the circumferential gap of the open-arc body over a selected serration 111 of the plunger rod 104, in a direction transverse to the longitudinal axis of the plunger rod 104—that is, laterally. The pressing force spreads the gap opening from its natural width of 3.03 mm to a width sufficient to accommodate the serration crest outer diameter of 4.25 mm as the gap passes over the crest of the selected serration, after which the elastic stainless steel body springs back, reducing the gap width to 3.03 mm and securing the clip 105 on the plunger rod 104. The inward tabs A, B, C now engage the serration valley of the selected serration, holding the clip 105 at the chosen dose-setting axial position.

The axial position of the clip 105 on the serrated plunger rod determines the maximum distance by which the plunger rod can be withdrawn proximally relative to the clip 105: as the plunger rod 104 is withdrawn, the clip 105 is held stationary by the outward tabs against the barrel inner wall, and withdrawal is limited when the proximal-most serration that the clip can reach abuts the inward tabs. This directly determines the maximum volume of fluid that can be aspirated into the barrel. By selecting different axial positions along the serrated region, different nominal fill volumes can be set using the same barrel and plunger rod design. For the barrel of the present invention with a 4.70 mm inner diameter, each serration pitch of 1.50 mm corresponds to a fluid volume of approximately 0.026 mL, compatible with the standard fixed vaccine volumes of 0.05 mL, 0.1 mL, 0.2 mL, 0.3 mL, and 0.5 mL.

Referring to FIG. 3B, a second assembly step S2 comprises inserting the plunger rod 104 carrying the locking clip 105 into the barrel 102 through the proximal opening. As the plunger rod and clip sub-assembly is advanced distally into the barrel, the outward tabs D and E of the locking clip 105 engage the inner wall of the barrel 102. The 15 degree proximal inclination of the outward tabs causes the tabs to deflect inwardly upon entry, permitting distal passage of the clip 105 into the barrel interior. Once the outward tabs have cleared the proximal opening and are seated at P1 within the barrel 102, the tab tips bear against the barrel inner wall in the proximal direction, preventing any proximal withdrawal of the locking clip 105. From this point forward, locking clip 105 is constrained to move only in the distal direction within barrel 102.

This anti-backout configuration of the clip 105 within the barrel 102 provides an important secondary benefit: if the assembled syringe 100 is subjected to vibration during transportation and logistics handling, the clip 105 cannot migrate proximally on the plunger rod 104 or within the barrel 102. The dose-setting position established in Step 1 is therefore preserved from assembly through to the point of use, ensuring that the nominal fill volume remains as set at manufacture regardless of shipping conditions. This addresses the transport stability requirement of ISO 7886-3:2020, Clause 13.5.

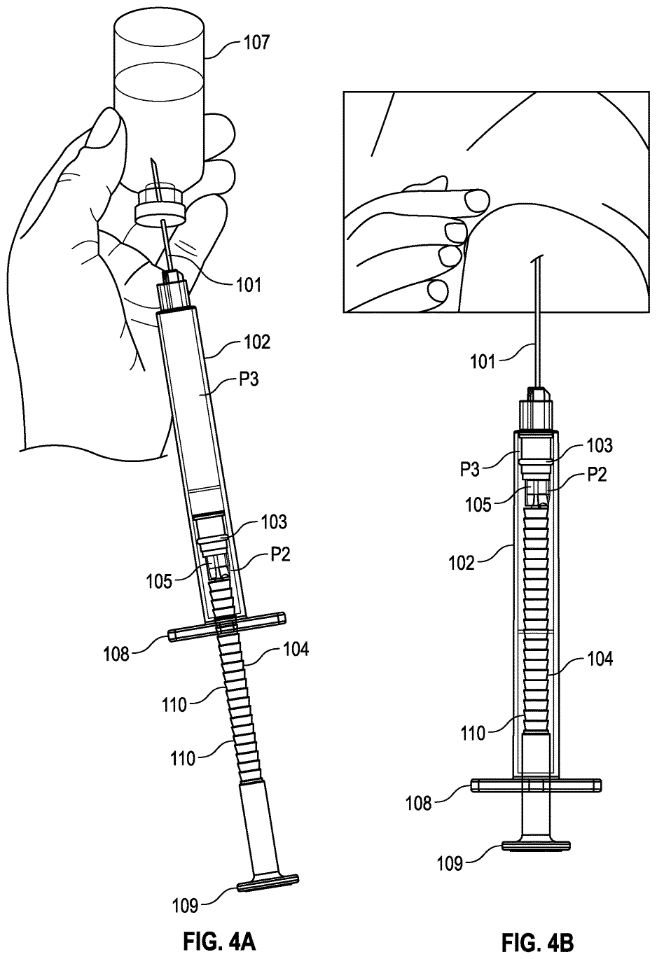

Referring to FIG. 4A, the assembled syringe 100 is provided to the user, such as a healthcare worker, with the needle cap 106 in place. To fill the syringe 100 with the nominal dose of vaccine, the healthcare worker removes the needle cap 106, inserts the needle 101 into the vaccine vial 107, and draws back the plunger rod 104 proximally by pulling the plunger rod flange 109.

As the plunger rod 104 is withdrawn proximally, the piston 103 moves proximally within the barrel 102, creating a reduced pressure that draws vaccine fluid from the vial 107 through the needle 101 into the barrel 102. During this proximal movement, the inward tabs A, B, C of the locking clip 105 flex outwardly over the sloped proximal faces of each successive serration, allowing the plunger rod 104 to slide proximally relative to the clip 105. The clip 105 is maintained in a fixed axial position within the barrel 102 by the outward tabs D and E, which resist proximal movement of the clip 105 by bearing against the inner wall of the barrel 102.

The proximal travel of the plunger rod 104 is limited when a distal end flange inner surface 112 of plunger rod 104 contacts the stationary distal flat face 113 of the clip. corresponding to the dose-setting position set at manufacture. At this stage, further proximal withdrawal of the plunger rod 104 is not possible. The volume of fluid aspirated corresponds to the nominal fill volume set by the clip position plus approximately 20% for air and dose adjusting, and the healthcare worker cannot aspirate more than this preset volume in a single fill stroke. While the needle remains in the fluid within the vial, the healthcare worker advances the plunger 104 distally to expel the excess fluid and air to precisely set the piston fiducial line with the scale dose mark. The syringe 100 is then ready for injection.

Referring to FIG. 4B, to administer the vaccine injection, the healthcare worker inserts the needle 101 into the patient and advances the plunger rod 104 distally by pressing on the plunger rod flange 109 with the thumb.

As the plunger rod 104 advances distally, the inward tabs A, B, C of the locking clip 105 engage the vertical distal face P2 of the serrations 110 and mechanically carry the locking clip 105 distally with the plunger rod 104. The outward tabs D and E flex slightly inwardly as the clip 105 is carried distally, allowing the clip 105 to travel through the barrel without generating excessive injection resistance. The three-point symmetric engagement of inward tabs A, B, C maintains the plunger rod 104 in coaxial alignment with the barrel axis throughout the injection stroke.

As the injection proceeds and the piston 103 approaches the fully distal position, the vaccine is expelled through the needle 101 into the patient. The clip 105 is carried distally P2 with the plunger rod 104 until the piston 103 has advanced to the full dose delivery position. At this point, the auto-disable mechanism has been full activated: the clip 105 is at its maximum distal position P2, and the outward tabs D and E are firmly embedded in the barrel inner wall at the distal end P3 of the barrel 102.

Following injection, if the healthcare worker attempts to withdraw the plunger rod 104 proximally, the distal clip flat surface 113 abuts up against the distal plunger rod flange 112 inner surfaces preventing proximal plunger/piston movement. However, the outward tabs D and E are now embedded in the barrel inner wall and resist proximal clip movement. The resultant axial tensile force is transferred through the clip engagement with the plunger rod distal flange 112 to the plunger rod frangible section. When the proximally directed force on the plunger rod 104 exceeds the fracture force of the frangible section 111 (38 to 55 Newtons), the frangible section 111 fractures, physically separating the proximal portion of the plunger rod 104 from the distal portion and piston 103. The syringe 100 is thereby rendered permanently and irreversibly unusable.

At no stage of this process is any additional action required of the user to activate the auto-disable feature. The feature is passively and automatically activated immediately upon starting of dose delivery, in full compliance with ISO 7886-3:2020, Clause 3.1 and Clause 13.4: “The syringe and needle shall be passively and automatically rendered unusable by the delivery of the intended fixed dose. No secondary or additional action on the part of the user shall be required.” This invention deploys “early activation” and continuous passive activation rather than the less preferred after the full dose is completed, which is the preferred method of WHO and UNICEF.

The auto-disable syringe 100 of the present invention is designed to conform in all respects to ISO 7886-3:2020. Table 2 below summarises the principal performance requirements and the corresponding design features of the present invention.

TABLE 2
ISO 7886-3: 2020 compliance summary for the auto-
disable syringe of the present invention.

| ISO 7886-3: 2020 | | |
|---|---|---|
| requirement | Clause | Compliance in the present invention |
| Barrel length shall avoid | 5(c) | Proximal barrel reduction of ~27% eliminates |
| unnecessary waste of | | non-functional barrel section. Fluid chamber |
| vaccine | | length 53.54 mm provides full dose delivery and |
| | | air expulsion. |
| Auto-disable shall be | 13.4 | Clip 105 automatically carried to locked distal |
| passively activated; no | | position during injection. No user action |
| user action required | | required. |
| Syringe shall not be | Annex C, | Frangible section fractures at 38-55 N, well |
| reusable under 100 N | C.3.1 | below 100 N threshold. |
| plunger force | | |
| Syringe shall not be | Annex C, | Clip embedded in barrel wall at full distal |
| reusable under 300 kPa | C.3.2 | position; piston cannot be driven back by back |
| back pressure | | pressure. |
| Maximum usable | 10.1 | Clip position and barrel length jointly limit |
| capacity not more than | | maximum fill to nominal dose + 25% or less. |
| 125% of nominal | | |
| Dead space: not more | Table 1 | Fixed integrated needle minimises dead space. |
| than 0.07 mL for 0.5 mL | | Confirmed by testing to be less than or equal to |
| nominal (integrated | | 0.02 mL |
| needle) | | |
| Plunger projection min. 8 | 11.1 | Plunger rod length 59.9 mm in 60.00 mm barrel |
| mm above barrel flange | | provides min. 8 mm projection at zero |
| at zero line | | graduation line. |
| Performance after | 13.5 | Clip anti-backout design (outward tabs resist |
| shipping vibration and | | proximal movement from the time of assembly) |
| drop tests | | prevents clip migration during transport. |

While the foregoing detailed description sets forth specific dimensions, materials, angular relationships, and operating forces with respect to the preferred embodiments, it will be understood by persons skilled in the art that the invention is not limited to these specific values. Various modifications, substitutions, and equivalents will be apparent to those skilled in the art without departing from the spirit and scope of the invention as defined by the appended claims. The embodiments described herein are illustrative and not restrictive. Where ranges of values are given, all values within those ranges and the endpoints thereof are intended to be encompassed by the claims. Reference to any prior art in this specification does not constitute an admission that such prior art forms part of the common general knowledge in the field, or that it could reasonably be expected to have been understood, regarded as relevant, or combined with other prior art by a person skilled in the art.

What is claimed is:

1. A fixed-dose auto-disable syringe comprising:
(a) a barrel formed from a transparent polymeric material and having a proximal end, a distal end, a barrel flange at the proximal end, an inner wall defining a fluid chamber, and a needle hub at the distal end;
(b) a plunger rod received within the barrel for axial movement therein, the plunger rod having a plunger rod flange at its proximal end and a piston at its distal end, the piston forming a fluid-tight sliding seal with the inner wall of the barrel, the plunger rod having a plurality of serrations on its outer surface; and
(c) an auto-disable mechanism configured to passively and automatically prevent reuse of the syringe upon delivery of a nominal dose volume, without any additional action by the user;
wherein the barrel has a reduced axial fluid chamber length, said reduction being achieved only by removal of a proximal barrel portion that does not contribute to the fluid chamber volume or to the plunger stroke length required for activation of the auto-disable mechanism, and wherein the inner diameter of the barrel, the distal barrel geometry, and the needle tip interface of the barrel are unchanged.

2. The syringe of claim 1, wherein the barrel has an axial fluid chamber length of between 48 mm and 60 mm and an inner diameter of between 4 mm and 6 mm.

3. The syringe of claim 1, wherein the ratio of the axial fluid chamber length of the barrel to the inner diameter of the barrel is between 8:1 and 14:1.

4. The syringe of claim 1, wherein the auto-disable mechanism comprises a locking clip mounted on the serrated plunger rod within the barrel, the locking clip having a body formed from a stamped metallic blank as an open arc defining a circumferential gap.

5. The syringe of claim 4, wherein the locking clip comprises:
(a) three inwardly directed tabs arranged at substantially equal angular intervals around the arc of the clip body, each of the three inwardly directed tabs being configured to engage the serrations on the plunger rod to permit distal movement of the clip relative to the plunger rod while resisting proximal movement of the clip relative to the plunger rod; and
(b) two outwardly directed tabs configured to engage the inner wall of the barrel to resist proximal movement of the clip relative to the barrel upon completion of the injection stroke;
wherein the three inwardly directed tabs are symmetrically arranged to maintain the plunger rod in coaxial alignment with the central axis of the barrel throughout the aspiration and injection strokes.

6. The syringe of claim 5, wherein the three inwardly directed tabs are evenly spaced at angular intervals of approximately 85 degrees around the arc of the clip body.

7. The syringe of claim 5, wherein each of the three inwardly directed tabs is inclined at an angle of between 8 degrees and 12 degrees relative to the longitudinal axis of the plunger rod, directed inwardly toward the plunger rod axis and toward the distal end of the syringe.

8. The syringe of claim 5, wherein each of the two outwardly directed tabs is inclined at an angle of between 13 degrees and 17 degrees relative to the longitudinal axis of the plunger rod, directed outwardly away from the plunger rod axis and toward the proximal end of the syringe.

9. The syringe of claim 5, wherein the two outwardly directed tabs are separated by an angular spacing of approximately 52 degrees.

10. The syringe of claim 4, wherein the open arc of the clip body subtends between 240 degrees and 270 degrees about the central axis of the plunger rod, defining the circumferential gap at an angle of between 90 degrees and 120 degrees, and wherein the width of the circumferential gap is less than the outer diameter of the serration crests of the plunger rod.

11. The syringe of claim 4, wherein the circumferential gap of the clip body is configured to permit placement of the locking clip onto the serrated plunger rod by pressing the gap over a serration of the plunger rod in a direction transverse to the longitudinal axis of the plunger rod.

12. The syringe of claim 4, wherein the locking clip is formed from a single flat sheet of stainless steel having a blank thickness of between 0.15 mm and 0.25 mm.

13. The syringe of claim 4, wherein the axial position of the locking clip on the serrated plunger rod at the time of manufacture determines the maximum volume of fluid aspiratable into the barrel through the needle upon proximal withdrawal of the plunger rod.

14. The syringe of claim 1, wherein the plunger rod has an axial length that is reduced in proportion to the reduction in axial fluid chamber length of the barrel.

15. The syringe of claim 1, wherein the piston is integrally formed as a unitary moulded component with the plunger rod.

16. A locking clip for an auto-disable syringe, the clip comprising:
(a) a clip body formed from a single flat sheet of stainless steel as an open arc subtending between 240 degrees and 270 degrees, defining a circumferential gap of between 90 degrees and 120 degrees having a gap opening width configured to permit lateral placement of the clip onto a plunger rod of a syringe in a direction transverse to the longitudinal axis of the plunger rod;
(b) three inwardly directed tabs arranged at substantially equal angular intervals around the arc of the clip body, each of the three inwardly directed tabs being inclined at between 8 degrees and 12 degrees relative to the central axis of the arc, configured to engage serrations on the plunger rod and to permit distal movement of the clip relative to the plunger rod while resisting proximal movement; and
(c) two outwardly directed tabs, each inclined at between 13 degrees and 17 degrees relative to the central axis of the arc, configured to engage an inner wall of the barrel of the auto-disable syringe to resist proximal movement of the clip relative to the barrel;
wherein the three inwardly directed tabs are arranged to maintain the plunger rod in coaxial alignment with the barrel axis of the syringe when the clip is assembled within the syringe.

17. The locking clip of claim 16, wherein the gap opening width is between 2.7 mm and 3.3 mm, the gap opening width being less than the serration root outer diameter of the plunger rod.

18. The locking clip of claim 16, wherein the stainless steel sheet has a blank thickness of between 0.15 mm and 0.25 mm.

19. The locking clip of claim 16, wherein the two outwardly directed tabs are separated by an angular spacing of approximately 52 degrees.

20. The locking clip of claim 16, wherein the three inwardly directed tabs are evenly spaced at angular intervals of approximately 85 degrees around the arc of the clip body.
